(19) United States
(12) Patent Application Publication
(10) Pub. No.: US 2006/0088494 A1
(43) Pub. Date: Apr. 27, 2006

(54) Method of treating hair with form memory polymers

(75) Inventors: Andreas Lendlein, Berlin (DE); Werner Grasser, Augsburg (DE); Karl Kratz, Moenchengladbach (DE); Thomas Krause, Darmstadt (DE); Axel Kalbfleisch, Darmstadt (DE); Gunther Lang, Reinheim (DE); Jurgen Allwohn, Burgschwalbach (DE); Johannes Burghaus, Gross-Umstadt (DE); Gerhard Sendelbach, Darmstadt (DE); Detlef Mattinger, Bickenbach (DE); Stefan Uhl, Darmstadt (DE)

Correspondence Address: KALOW & SPRINGUT LLP, 488 MADISON AVENUE, 19TH FLOOR, NEW YORK, NY, 10022, US

(73) Assignee: Mnemoscience GMBH, Uebach-Palenberg (DE)

(21) Appl. No.: 10/510,873

(22) PCT Filed: Apr. 10, 2003
(86) PCT No.: PCT/EP03/03735

§ 371 Date: Sep. 12, 2005

(30) Foreign Application Priority Data
Apr. 10, 2002 (DE) 102 15 858.4

(51) Int. Cl. A61K 8/81

(52) U.S. Cl. 424/70.16

(57) ABSTRACT

A method for the provision of a recallable hair transformation using shape-memory polymers is disclosed. In this respect a composition which comprises a macromer or prepolymer which is cross-linkable to a shape-memory polymer is applied onto the hair and the hair is brought into a defined (permanent) shape and subsequently the permanent shape is fixed by means of the chemical cross-linking of the macromer or prepolymer under formation of the shape-memory polymer. The macromer or prepolymer contains segments which are cross-linkable by means of chemical bonds as well as thermoplastic segments which are not chemically cross-linkable. The shape-memory polymers do possess at least one transition temperature T_{Trans}. In addition to the permanent shape of a hairdo a second (temporary) shape can be impressed. Furthermore, disclosed is a method for the recovery of a previously programmed, permanent shape of a hairdo, which has been temporarily brought into a temporary form or which has been deformed in a different manner.

In the field of shaping of hair, a distinction is usually made between temporary hair-shaping methods and long-lasting, permanent hair-shaping methods. A temporary hair-shaping usually occurs in connection with the use of compositions on the basis of solutions or dispersions comprising hair-fixative polymers. Such products provide the hair, by means of the polymers used, with improved hold, volume, elasticity, body and shine. Such styling products facilitate, for example, in the form of a gel, the shaping and provision of a hairdo; they improve, in the form of hair spray, the condition of a prepared hairdo; and they increase, in the form of setting foams, the volume of the hair. A drawback in this connection is that the desired effects are of only relatively short duration, and that they are lost under external influences, such as combing, wind, high humidity, or contact with water. A permanent hair transformation usually occurs during a treatment of providing a permanent wave. During this treatment the disulfide bonds in the hair are cleaved by use of a reductive reaction, the hair is brought into the new shape, and this new shape is fixed by providing new disulfide bonds by means of an oxidative process. A drawback in this respect, however, is that the required chemical treatment of the hair employing reduction agents and oxidation agents involves an impairment of the structure of the hair. A further drawback of the methods for hair shaping known up to now is that it is not possible to revert a once-performed transformation (shaping) using relatively simple means, i.e., it is not possible to change from one shape of a hairdo to another shape of a hairdo without troublesome processes.

JP 04-41416 discloses compositions in the field of cosmetic preparations for hair which comprise specific linear polyurethanes having a glass transition temperature T_{g} of from 40 to 90° C. The process for hair treatment disclosed therein corresponds to the treatment using conventional thermoplastic materials. After the administration of the composition, a hairdo is prepared at a temperature above T_{g}, and this hairdo is then fixed by means of cooling under T_{g}. During a subsequent rewarming to a temperature above T_{g}, the polymer softens and a new hairdo can be prepared. A method for a recallable, reversible hair-shaping (hair transformation), however, is not disclosed. The properties of the linear polyurethanes are not satisfactory in order to provide a recallable hair shaping.

The object underlying the present invention was to provide a method and the therefore required products for providing a recallable hair transformation (hair-shaping) enabling a high degree of recovery of a once-programmed hairdo. A further object was to provide a method enabling a permanent hair-shaping without requiring an impairing intervention into the hair structure. A further object was the provision of a method which enables the reversion of a temporary transformation/shaping over multiple cycles with a high accuracy in order to revert to a previously prepared, programmed permanent hairdo. A further object was also the provision of a method which enables in a simple manner to remedy with a high accuracy the deformation of a hairdo which has been introduced by external influences, in order to revert to a once prepared, programmed permanent hairdo.

This object is solved with a method for hair treatment, comprising the steps of

applying a composition, which comprises at least one crosslinkable macromer, which forms after crosslinking a shape-memory polymer, onto the hair, wherein the macromer
a. comprises crosslinkable areas (segments) which are crosslinkable by means of chemical bonds and
b. thermoplastic areas (segments) which are not chemically crosslinkable,
bringing the hair, previously, at the same time or subsequently, into a desired (permanent) shape and
fixing the shape by means of chemical crosslinking of the macromer, thereby providing the shape memory polymer,

characterized in that the shape memory polymer possesses at least one transition temperature Tₜᵣₐₙₛ.

A further object of the present invention is a method for impressing a second hairdo over a programmed, recallable first hairdo. In this method, a hairdo, programmed by means of the above-explained method (permanent shape) is heated to a temperature above Tₜᵣₐₙₛ. Subsequently, the hair is brought into the second (temporary) shape and the second shape is fixed by means of cooling to a temperature below Tₜᵣₐₙₛ.

A further subject of the present invention is a method for the recovery of a first hairdo, programmed by means of the above-explained method (permanent shape). Therein, a hairdo in a temporary shape or in the shape of a hairdo obtained by means of cold forming, is heated to a temperature above Tₜᵣₐₙₛ.

Shape-memory polymers in accordance with the present invention are polymers from which materials can be prepared which possess the property that they can be impressed with a desired shape (permanent shape), to which these materials, after deformation or after impressing a second shape (temporary shape), can revert spontaneously and without external forces by means of simple warming or by means of another energetic stimulus. Deformation and retransformation (recovery) can be carried out over multiple cycles. The degree of attainability of the initial, permanent shape, is during a first relaxation cycle consisting of deformation and recovery, usually somewhat smaller than during the subsequent cycles, most probably due to the removal of existing textures, defects, etc., initially present. A very high degree of recovery will, however, be attained during the subsequent relaxation cycles. The degree of recovery during the first relaxation cycle is preferably at least 30%, in particular at least 50% and the degree of recovery during the subsequent relaxation cycles is preferably at least 60%, more preferably at least 80%. This degree may, however, be also 90% or more. The degree of recovery can be measured in accordance with usual retention measurements by means of the determination of the length of a treated hair strand or by means of known, suitable stress-strain experiments.

Macromers or pre-polymers in accordance with the present invention which can be crosslinked in order to provide shape-memory polymers, are polymers or oligomers wherein the fixation of an impressed, permanent shape occurs by means of chemical bonds connecting specific polymer strands or oligomer strands. The crosslinkage, by means of chemical bonds, can be provided by means of ionic or covalent bonds. The crosslinking reaction may be any suitable chemical reaction, for example, a salt formation reaction, a condensation reaction, an addition reaction, a substitution reaction or a reaction initiated photochemically or by means of a radical. The crosslinking reaction can occur using suitable catalysts or initiators or the crosslinking reaction can occur without the use of a catalyst. The crosslinking reaction can be initiated by means of a suitable energy source, for example, electromagnetic radiation, ultrasound, heat or mechanical energy. A combination of two or more methods for initiation can be employed in order to increase the efficiency or the velocity of the crosslinking reaction.

Shape-memory polymers which can be used in accordance with the present invention possess at least one transition temperature Tₜᵣₐₙₛ. This transition temperature may be a melting temperature Tₘ or a glass transition temperature T_{g}. Above Tₜᵣₐₙₛ, the polymer has a lower modulus of elasticity than below Tₜᵣₐₙₛ. The ratio of the modulus of elasticity below Tₜᵣₐₙₛ to above Tₜᵣₐₙₛ is preferably at least 20. The transition temperature Tₜᵣₐₙₛ is preferably above room temperature (20° C.), preferably this transition temperature is at least 30° C., in particular preferred 40° C. The transition temperature Tₜᵣₐₙₛ is the temperature above which the spontaneous recovery of the permanent shape, starting from the deformed shape or the temporary shape, occurs.

The terms “hairdos” or “shape of hair” in accordance with the present invention are to be understood broadly and comprise in particular also the degree of waviness or the degree of evenness of hair. A programmed hairdo in accordance with the present invention is any group of hairs which show a specific shape due to shape-memory polymers which have been crosslinked and which fix a permanent shape. Recovery of a programmed hairdo in accordance with present invention means that the programmed hairdo is recovered after a deformation, to a degree of at least 60%, preferably to a degree of at least 80%, in relation to the shape which can be obtained after a first relaxation cycle. The degree of recovery can, for example, be determined by means of the determination of the length of a hair strand or hair curl.

Suitable macromers or pre-polymers, which can be chemically crosslinked in order to provide shape-memory polymers are macromonomers which can be polymerized or which can be crosslinked by means of single chemical bonds. The chemically crosslinkable polymers are designated as thermoset polymers in WO 99/42147. The macromers and thermoset polymers disclosed in WO 99/42147 are suitable for use in accordance with the present invention and these materials are incorporated here by reference. Soft, thermoplastic segments (switching segments) having a transition temperature Tₜᵣₐₙₛ are crosslinked by means of chemical, preferably covalent bonds. Required are switching segments and fixation points (network points). The fixation points fix the permanent form while the switching segments fix the temporary shape. The shape memory effect is based on the change of the elasticity at, above, or below Tₜᵣₐₙₛ or during the transition from above to below Tₜᵣₐₙₛ or from below to above Tₜᵣₐₙₛ. The ratio of the modulus of elasticity below Tₜᵣₐₙₛ to above Tₜᵣₐₙₛ is preferably at least 20. The higher this ratio is, the more expressed the shape-memory effect can be obtained. Four types of thermoset polymers having shape-memory properties can be designated:

Network polymers, penetrating network polymers, semi-interpenetrating networks and mixed penetrating networks. Network polymers can be formed by means of the covalent connection of macromonomers, i.e., of oligomers or polymers having reactive terminal groups, preferably ethylenically unsaturated terminal groups, terminal groups which can be reacted by means of radical reactions or terminal groups which can be reacted by means of photochemical reactions. The crosslinking reaction can, for example, be initiated by means of light-sensitive or temperature-sensitive initiators, by means of red-ox systems or combinations thereof, or the reaction can be initiated without the use of initiators, e.g., using UV light, heat or mechanical energy. Interpenetrating networks are formed at least two components which are each crosslinked but not with the other component. Mixed interpenetrating networks are formed using at least two components, wherein one component is crosslinked by means of chemical bonds and the second component is crosslinked by means of physical interactions. Semi-interpenetrating networks are formed using at least two components, of which one is chemically crosslinkable, while the other one is not chemically crosslinkable. Both components, however, cannot be separated from one another by means of physical methods.

In principle, all synthetic or natural oligomers or polymers having reactive terminal groups or side chain groups, which provide the crosslinked shape-memory polymer with a suitable transition temperature Tₜᵣₐₙₛ and suitable moduli of elasticity above and below Tₜᵣₐₙₛ are suitable, wherein the terminal groups or the side chain groups are already present initially or are provided by means of a subsequent derivatization. These materials allow a crosslinking reaction using the above-identified methods. Suitable macromers are, for example, macromers having the following formula
A1-(X)n-A2 (I)
wherein A1 and A2 designate reactive, chemically crosslinkable groups and wherein

—(X)n- designates a divalent, thermoplastic polymer or oligomer segement. A1 and A2 are preferably acrylate or methacrylate groups. The segment —(X)n- preferably designates a polyester segment, an oligoester segment, a polyalkylene glycol segment, an oligoalkylene glycol segment, a polyalkylene carbonate segment or a oligoalkylene carbonate segment, wherein the alkylene groups are preferably ethylene groups or propylene groups. Suitable macromonomers for the formation of thermoset polymers having shape-memory properties are oligo-(ε-caprolactone)or poly(ε-caprolactone), oligolactide or polylactide, oligoalkylene glycol, olyalkyleneglycol, e.g., polyethylene glycol or their block copolymers, wherein the polymers or oligomers possess at least two ethylenically unsaturated groups, which can be polymerized by means of a radical reaction, e.g., acrylates or methacrylates, wherein these groups are provided at a terminal position or at any side chain position.

The polymer segments can be chosen among natural polymers, such as, for example, segments derived from proteins or polysaccharides. Suitable are also synthetic polymer segments. Suitable natural polymer segments are proteins such as zein, modified zein, casein,gelatine, gluten, serum albumin or collagen, as well as polysaccharides such as alginate, cellulose, dextrane, pullulane or polyhyaluronic acid, as well as chitin, poly(3-hydroxyalkanoate), especially poly(ε-hydroxybutyrate), poly(3-hydroxyoctanoate) or poly(3-hydroxyfatty acids). Suitable are also derivatives of natural polymer segments such as alkylated, hydroxyalkylated, hydroxylated or oxidated modifications.

Synthetically modified natural polymers are, for example, cellulose derivatives such as alkylcelluloses, hydroxyalkylcelluloses, cellulose ethers, cellulose esters, nitrocellulose, chitosan or chitosan derivatives, which are, e.g., obtained by means of alkyl substitution or hydroxyalkyl substitution at the nitrogen and/or oxygen. Examples are methylcellulose, ethylcellulose, hydroxypropylcellulose, hydroxypropylmethylcellulose, hydroxybutylmethylcellulose, celluloseacetate, cellulosepropionate, celluloseacetatebutyrate, celluloseacetatephthalate, carboxymethylcellulose, cellulosetriacetate or cellulosesulfate sodium salt. These materials are all designated in the following as “celluloses.”

Suitable synthetic polymer blocks include polyphosphazenes, poly(vinylalcohols), polyamides, polyesteramides, poly(aminoacids), polyanhydrides, polycarbonates, polyacrylates, polyalkylenes, polyacrylamides, polyalkylenglycoles, polyalkylenoxides, polyalkylenterephthalates, polyorthoesters, polyvinylethers, polyvinylesters, polyvinylhalogenides, polyvinylpyrrolidones, polyesters, polylactide, polyglycolide, polysiloxanes, polyurethanes and copolymers thereof. Examples of suitable polyacrylate are poly(methylmethacrylate), poly(ethylmethacrylate), poly(butylmethacrylate), poly(isobutylmethacrylate), poly(hexylmethacrylate), poly(isodecylmethacrylate), poly(laurylmethacrylate), poly(phenylmethacrylate), poly(methylacrylate), poly(isopropylacrylate), poly(isobutylacrylate) or poly(octadecylacrylate). Suitable synthetic, biologically degradable polymer segments are polyhydroxyacids such as polylactide, polyglycolide and and copolymers thereof, poly-(ethylenterephthalate); poly(hydroxybuturic acid); poly-(hydroxyvaleric acid); poly[lactide-co-(ε-caprolactone)]; poly[glycolide-co-(ε-caprolactone)]; polycarbonates, poly(aminoacids); poly(hydroxyalkanoates); polyanhydrides; polyorthoesters as well as mixtures and copolymers thereof. Examples of polymer segments which are less readily biologically degradable are poly(methacrylic acid), poly(acrylic acid), polyamides, polyethylene, polypropylene, polystyrene, polyvinylchloride, polyvinylphenol as well as mixtures and copolymers thereof.

Compositions in accordance with present invention for the treatment of hair comprise at least one of the above-mentioned macromers or pre-polymers, which is suitable to be crosslinked in order to provide shape-memory polymers, in an amount of preferably from 0.01 to 25 weight %, more preferably of from 0.1 to 15 weight % in a suitable liquid medium. The composition can be provided in the form of a solution, dispersion, emulsion, suspension or latex. The liquid medium is preferably cosmetically acceptable and physiologically acceptable.

In a preferred embodiment of the present invention, the composition comprises a mixture of (A) macromers which are substituted with at least two reactive, crosslinkable groups, and (B) macromers, which are substituted with only one reactive group. Suitable additional macromers are, for example, macromers of the following general formula:
R—(X′)ₙ-A3 (II)

wherein R designates a monovalent organic residue, A3 designates a reactive, chemically crosslinkable group and —(X′)ₙ designates a divalent, thermoplastic polymer segment or oligomer segment. A3 preferably represents an acrylate group or a methacrylate group. The segment —(X′)ₙ— preferably represents a polyalkyleneglycol, monoalkylethers thereof, or block copolymers thereof, wherein the alkylene groups preferably are ethylene or propylene groups and wherein the alkyl groups preferably comprise from 1 to 30 carbon atoms.

Particularly preferred are mixtures of (A) polyalkyleneglycoles or polycaprolactones having terminal ester groups comprising acrylic acid or methacrylic acid and (B) polyalkyleneglycol-monoalkylethers, having one terminal ester group comprising acrylic acid or methacrylic acid wherein the alkylene groups are preferably ethylene or propylene groups and the alkyl groups are preferably C1- bis C30-alkyl groups.

As component (A) the following are, e.g., suitable: poly(ε-caprolactone)-dimethacrylate, poly(DL-lactide)-dimethacrylate, poly(L-lactide-co-glycolide)-dimethacrylate, poly(ethyleneglycol)dimethacrylate, poly(propylene-glycol)dimethacrylate, PEG-block-PPG-block-PEG-dimethacrylate, poly(ethyleneadipate)-dimethacrylate, hexamethylencarbonat-dimethacrylate.

Suitable as component (B) are, e.g., the following: poly(ethyleneglycol)monoacrylate, poly(propyleneglycol)monoacrylate and monoalkylethers thereof.

The composition in accordance with present invention usually is present in the form of a solution or dispersion comprising a suitable solvent. Preferred are in particular aqueous, alcoholic or aqueous-alcoholic solvents. Suitable solvents are, e.g., aliphatic alcohols having from 1 to 4 carbon atoms or a mixture of water with at least one of these alcohols. Other organic solvents can, however, also be employed, in particular linear or branched hydrocarbons, such as pentane, hexane, isopentan, cyclic hydrocarbons such as cyclopentane and cyclohexan, organic linear or cyclic ethers, such as tetrahydrofurane (THF) or liquid organic esters, such as ethylacetate. Furthermore, solvents on the basis of silicone materials are suitable, in particular silicone oils on the basis of linear or cyclic polydimethylsiloxanes (dimethicone or Cyclomethicone). The solvents are preferably present in an amount from 0,5 to 99 weight %, more preferably in an amount from 40 to 90 weight %.

The compositions in accordance with present invention may comprise in addition from 0.01 to 25 weight % of at least one hair fixative, hair coloring agent and/or hair cosmetic. Hair fixatives are in particular the known conventional film-forming hair fixative polymers. The film forming and hair fixative polymer may be of synthetic origin or of natural origin and may be of nonionic, cationic, anionic or amphoteric character. Such a polymer additive, which can be present in an amount of from 0.01 to 25 weight %, preferably 0.1 to 20 weight %, more preferably 0.5 to 15 weight %, may also comprise a mixture of more than one polymers, and this additive can be modified further with respect to the hair fixative properties by means of the addition of further polymers having a thickening effect. Film-forming, hair fixative polymers in accordance with present invention are polymers which are able, when employed in aqueous, alcoholic or aqueous-alcoholic solutions at a concentration of from 0.01 to 5%, to provide a polymer film on the hair in order to provide fixative properties.

As suitable, synthetic, nonionic, film-forming, hair fixative polymers, the hair treatment composition in accordance with present invention may comprise homo polymers of vinyl pyrrolidone, homopolymers of N-vinylformamide, copolymers of vinylpyrrolidone and vinylacetate, terpolymers of vinylpyrrolidone, vinylacetate and vinylpropionate, polyacrylamide, polyvinylalcohols, or polyethyleneglycols having a molecular weight of from 800 to 20.000 g/mol. Suitable, synthetic, anionic, film-forming polymers are crotonic acid/vinylacetate copolymers and terpolymers of acrylic acid, ethylacrylate and N-t-butylacrylamide. Natural film-forming polymers or polymers obtained therefrom by means of chemical derivatization may also be employed in the hair treatment composition in accordance with present invention. In this respect low molecular chitosane having a molecular weight of from 30,000 to 70,000 g/mol or high molecular chitosane, derivatives of chitosane soluble in organic solvents, mixtures of oligosaccharides, monosaccharides, and disaccharides, China balsam resin, cellulose derivatives such as hydroxypropylcellulose having a molecular weight of from 30,000 to 50,000 g/mol, or shellac in its neutralized or unneutralized form are known as suitable. Amphoteric polymers can also be employed in the hair treatment composition in accordance with present invention. Suitable are, e.g., copolymers of octylacrylamide, t-butylaminoethylmethacrylate and two or more monomers selected from the group consisting of acrylic acid, methacrylic acid and simple esters thereof.

Cationic polymers which can be used in accordance with present invention comprise copolymers of vinylpyrrolidones with quaternized derivatives of dialkylaminoacrylate and dialkylaminomethacrylate, such as, for example, the copolymer of vinylpyrrolidone and dimethylaminomethacrylate quaternized with diethylsulfate. Further cationic polymers are copolymers of vinylpyrrolidone with vinylimidazoliummethochloride, the terpolymer of dimethyldiallylammoniumchloride, sodiumacrylate and acrylamide, the terpolymer of vinylpyrrolidone, dimethylaminoethylmethacrylate and vinylcarprolactam, the quaternized ammonium salt, prepared from hydroxyethylcellulose and a trimethylammonium substituted epoxide, the vinylpyrrolidone and methacrylamidopropyltrimethylammoniumchloride and diquaternized polydimethylsiloxanes.

The consistency of hair treatment compositions in accordance with present invention can be improved by means of the addition of thickeners. In this respect, homopolymers of acrylic acid having a molecular weight of 2,000,000 to 6,000,000 g/mol are suitable. Further copolymers of acrylic acid and acrylamide (sodium salt) having a molecular weight of from 2,000,000 to 6,000,000 g/mol, sclerotium gum and copolymers of acrylic acid and methacrylic acid are suitable.

A cosmetic composition in accordance with present invention can be employed in the form of different formulations for different types of applications, e.g., as lotion, as spray lotion, as cream, as gel, as foam-gel, as aerosol spray, as non-aerosol spray, as aerosol foam, as non-aerosol foam, as o/w- or w/o-emulsion, as micro emulsion or as hair wax.

When the hair treatment composition in accordance with present invention is provided in the form of an aerosol spray, the composition comprises additionally from 15 to 85 weight %, preferably from 25 to 75 weight % of a propellant and the composition is filled into a pressurized canister having a spray button As propellants, lower alkanes, such as n-butane, isobutene and propane as well as mixtures thereof as well as dimethylether or fluorohydrocarbons, such as F 152a (1,1-difluorethane) or F 134 (tetrafluorethane) are suitable, as well as propellants which are present at the pressures in question in gaseous forms such as N₂, N₂O and CO₂ as well as mixtures of the above-mentioned propellants.

When the hair treatment composition of the present invention is provided in the form of a sprayable non-aerosol hair spray, the composition is sprayed by means of a suitable, mechanical spraying device. Spray devices of the mechanical type are devices which allow the spraying of a composition without using a propellant. Suitable mechanical spray devices are, e.g., spray pumps or elastic containers provided with a spray valve, into which the cosmetic composition in accordance with present invention is filled under pressure, whereby the elastic container is expanded and wherein the cosmetic composition is continuously discharged if the valve is open, due to the contraction of the elastic container.

When the hair treatment composition in accordance with present invention is provided in the form of a hair foam (mousse), the composition comprises at least one conventional foaming agent known in the art for this purpose. The composition is foamed with or without the aid of propellants or chemical propellants and the foam may be applied to the hair and may remain in the hair without the need of rinsing the hair. A product in accordance with present invention comprises as additional component a device for the provision of a foam of the composition. Devices for providing a foam are devices which allow the provision of a foam starting from a liquid, with or without the use of a propellant. Suitable mechanical foaming devices are, e.g., usual foam pumps, or usual aerosol foaming heads.

When the hair treatment composition in accordance with present invention is provided in the form of a hair gel, it comprises at least one gel forming substance in an amount of preferably from 0.05 to 10, more preferably from 0.1 to 2 weight %. The viscosity of the gel preferably amounts to from 100 bis 50,000 mm²/s, more preferably 1,000 bis 15,000 mm²/s at 25° C., measured as dynamic viscosity using a Bohlin Rheometer CS, measurement body C25 using a shear velocity of 50 s⁻¹.

When the hair treatment composition in accordance with present invention is provided in the form of a hair wax, it comprises additionally water-soluble fatty substances or waxy substances, or substances which provide the composition with a waxy consistence, in an amount of preferably 0.5 bis 30 weight %. Suitable water unsoluble substances are, for example, emulgators having a HLB-value of below 7, silicone oils, silicone waxes, wax materials (e.g., waxy alcohols, waxy acids, waxy esters, as well as natural waxes such as beeswax, Carnauba wax, etc.), fatty alcohols, fatty acids, fatty acid esters or high molecular polyethyleneglycols having a molecular weight of from 800 bis 20,000, preferably from 2,000 to 10,000 g/mol.

When the hair treatment composition in accordance with present invention is provided in the form of a hair lotion, it is present as a substantially non-viscous or low viscosity, flowable solution, dispersion or emulsion comprising a content of at least 10 weight %, preferably 20 to 95 weight % of a cosmetically acceptable alcohol. As alcohols, the usual alcohols used for cosmetic purposes can be named, in particular the lower C1 to C4 alcohols such as ethanol and isopropanol.

When the hair treatment composition of the present invention is present in the form of a hair cream, it is preferably provided in the form of an emulsion and it either comprises additional viscosity providing components in an amount of from 0.1 to 10 weight % or the required viscosity and creamy consistency is obtained by means of micelle formation using suitable emulsifiers, fatty acids, fatty alcohols, waxes, etc.

In a preferred embodiment the composition in accordance with present invention is provided in a form enabling, at the same time, the impression of a recallable hairdo as well as providing hair coloration. The composition is then formulated as coloring hair treatment composition, e.g., as coloring fixative, coloring cream, coloring foam, etc. It comprises at least one coloring component. The coloring component may be an organic dye, in particular direct dyes or it may be an inorganic pigment.

The total amount of coloring agent amounts in the composition in accordance with present invention to about 0.01 to 7 weight %, preferably about 0.2 to 4 weight %. Suitable direct colorants to be used in the composition in accordance with present invention are, e.g., triphenylmethane colorants, aromatic nitro colorants, azo colorants, quinone colorants, cationic or anionic colorants. Suitable are the following:

Nitro dyes (blue):

1,4-bis[(2-hydroxyethyl)amino]-2-nitrobenzene, 1-(2-hydroxy-ethyl)amino-2-nitro-4-[di(2-hydroxyethyl)amino]-benzene (HC Blue No. 2), 1-amino-3-methyl-4-[(2-hydroxyethyl)amino]-6-nitrobenzene (HC Violet No. 1), 4-[ethyl-(2-hydroxyethyl)-amino]-1-[(2-hydroxyethyl)amino]-2-nitrobenzene-hydrochloride (HC Blue No. 12), 4-[di(2-hydroxyethyl)amino]-1-[(2-methoxy-ethyl)amino]-2-nitrobenzene (HC Blue No. 11), 1-[(2,3-di-hydroxypropyl)amino]-4-[methyl-(2-hydroxyethyl)amino]-2-nitrobenzene (HC Blue No. 10), 1-[(2,3-dihydroxypropyl)amino]-4-[ethyl-(2-hydroxyethyl)amino]-2-nitrobenzene-hydrochloride (HC Blue No. 9), 1-(3-hydroxypropylamino)-4-[di(2-hydroxyethyl)amino]-2-nitrobenzene (HC Violet No. 2), 1-methylamino-4-[methyl-(2,3-dihydroxypropyl)amino]-2-nitro-benzene (HC Blue No. 6), 2-((4-amino-2-nitrophenyl)amino)-5-dimethylamino-benzoic acid (HC Blue No. 13), 1-(2-aminoethyl-amino)-4-[di(2-hydroxyethyl)amino]-2-nitrobenzene, 4-(di(2-hydroxyethyl)amino)-2-nitro-1-phenylamino-benzene.

Nitro dyes (red):

1-amino-4-[(2-hydroxyethyl)amino]-2-nitrobenzene (HC Red No. 7), 2-amino-4,6-dinitro-phenene, 1,4-diamino-2-nitrobenzene (CI76070), 4-amino-2-nitro-diphenylamine (HC Red No. 1), 1-amino-4-[di(2-hydroxyethyl)amino]-2-nitrobenzene-hydrochloride (HC Red No. 13), 1-amino-5-chlor-4-[(2-hydroxyethyl)amino]-2-nitrobenzene, 4-amino-1-[(2-hydroxyethyl)amino]-2-nitrobenzene (HC Red No. 3), 4-((2-hydroxyethyl)methylamino)-1-(methylamino)-2-nitrobenzene, 1-amino-4-((2,3-dihydroxy-propyl)amino)-5-methyl-2-nitrobenzene, 1-amino-4-(methylamino)-2-nitrobenzene, 4-amino-2-nitro-1-((prop-2-en-1-yl)amino)-benzene, 4-amino-3-nitrophenene, 4-[(2-hydroxyethyl)amino]-3-nitrophenene, 4-[(2-nitrophenyl)amino]phenene (HC Orange No. 1), 1-[(2-aminoethyl)amino]-4-(2-hydroxyethoxy)-2-nitrobenzene (HC Orange No. 2), 4-(2,3-dihydroxypropoxy)-1-[(2-hydroxyethyl)amino]-2-nitrobenzene (HC Orange No. 3), 1-amino-5-chlor-4-[(2,3-dihydroxypropyl)amino]-2-nitrobenzene (HC Red No. 10), 5-chlor-1,4-[di(2,3-dihydroxypropyl)amino]-2-nitrobenzene (HC Red No. 11), 2-[(2-hydroxyethyl)amino]-4,6-dinitro-phenene, 4-ethylamino-3-nitrobenzoic acid, 2-[(4-amino-2-nitrophenyl)amino]-benzoic acid, 2-chlor-6-ethylamino-4-nitrophenene, 2-amino-6-chlor-4-nitrophenene, 4-[(3-hydroxypropyl)amino]-3-nitrophenene, 2,5-diamino-6-nitro-pyridine, 6-amino-3-((2-hydroxyethyl)amino)-2-nitropyridine, 3-amino-6-((2-hydroxyethyl)amino)-2-nitropyridine, 3-amino-6-(ethylamino)-2-nitropyridine, 3-((2-hydroxyethyl)amino)-6-(methylamino)-2-nitropyridine, 3-amino-6-(methylamino)-2-nitropyridine, 6-(ethylamino)-3-((2-hydroxyethyl)amino)-2-nitropyridine, 1,2,3,4-tetrahydro-6-nitrochinoxaline, 7-amino-3,4-dihydro-6-nitro-2H-1,4-benzoxazin (HC Red No. 14).

Nitro dyes (yellow):

1,2-diamino-4-nitrobenzene (CI76020), 1-amino-2-[(2-hydroxy-ethyl)amino]-5-nitrobenzene (HC Yellow No. 5), 1-(2-hydroxy-ethoxy)-2-[(2-hydroxyethyl)amino]-5-nitrobenzene (HC Yellow No. 4), 1-[(2-hydroxyethyl)amino]-2-nitrobenzene (HC Yellow No. 2), 2-(di(2-hydroxyethyl)amino)-5-nitrophenene, 2-[(2-hydroxyethyl)amino]-1-methoxy-5-nitrobenzene, 2-amino-3-nitrophenene, 1-amino-2-methyl-6-nitrobenzene, 1-(2-hydroxy-ethoxy)-3-methylamino-4-nitrobenzene, 2,3-(dihydroxypropoxy)-3-methylamino-4-nitrobenzene, 2-[(2-hydroxyethyl)amino]-5-nitrophenene (HC Yellow No. 11), 3-[(2-aminoethyl)amino]-1-methoxy-4-nitrobenzene-hydrochloride (HC Yellow No.9), 1-[(2-Ureidoethyl)amino]-4-nitrobenzene, 4-[(2,3-dihydroxypropyl)-amino]-3-nitro-1-trifluormethyl-benzene (HC Yellow No. 6), 1-chlor-2,4-bis[(2-hydroxyethyl)amino]-5-nitrobenzene (HC Yellow No. 10), 1-amino-4-((2-aminoethyl)amino)-5-methyl-2-nitrobenzene, 4-[(2-hydroxyethyl)amino]-3-nitro-1-methyl-benzene, 1-chlor-4-[(2-hydroxyethyl)amino]-3-nitrobenzene (HC Yellow No. 12), 4-[(2-hydroxyethyl)amino]-3-nitro-1-tri-fluormethyl-benzene (HC Yellow No. 13), 4-[(2-hydroxyethyl)-amino]-3-nitro-benzonitril (HC Yellow No. 14), 4-[(2-hydroxyethyl)amino]-3-nitro-benzamide (HC Yellow No. 15) 3-((2-hydroxyethyl)amino)-4-methyl-1-nitrobenzene, 4-chlor-3-((2-hydroxyethyl)amino)-1-nitrobenzene.

Quinone dyes:

1,4-di[(2,3-dihydroxypropyl)amino]-9,10-anthraquinone, 1,4-di[(2-hydroxyethyl)amino]-9,10-anthraquinone (CI61545, Disperse Blue 23), 1-[(2-hydroxyethyl)amino]-4-methylamino-9,10-anthraquinone (CI61505, Disperse Blue No. 3), 2-[(2-aminoethyl)amino]-9,10-anthraquinone (HC Orange No. 5), 1-amino-4-hydroxy-9,10-anthraquinone (CI60710, Disperse Red 15), 1-hydroxy-4-[(4-methyl-2-sulfophenyl)amino]-9,10-anthraquinone, 7-beta-D-glucopyranosyl-9,10-dihydro-1-methyl-9,10-dioxo-3,5,6,8-tetrahydroxy-2-anthracencarboxylic acid (CI75470, Natural Red 4), 1-[(3-aminopropyl)amino]-4-methylamino-9,10-anthraquinone (HC Blue No. 8), 1-[(3-aminopropyl)amino]-9,10-anthraguinone (HC Red No. 8), 1,4-diamino-2-methoxy-9,10-anthraquinone (CI62015, Disperse Red No. 11, Solvent Violet No. 26), 1,4-dihydroxy-5,8-bis[(2-hydroxyethyl)amino]-9,10-anthraquinone (CI62500, Disperse Blue No. 7, Solvent Blue No. 69), 1,4-diamino-9,10-anthraquinone (CI61100, Disperse Violet No. 1), 1-amino-4-(methylamino)-9,10-anthraquinone (CI61105, Disperse Violet No. 4, Solvent Violet No. 12), 2-hydroxy-3-methoxy-1,4-naphthoquinone, 2,5-dihydroxy-1,4-naphthoquinone, 2-hydroxy-3-methyl-1,4-naphthoquinone, N-(6-((3-chlor-4-(methylamino)phenyl)imino)-4-methyl-3-oxo-1,4-cyclohexadien-1-yl)urea (HC Red No. 9), 2-((4-(di(2-hydroxyethyl)amino)-phenyl)amino)-5-((2-hydroxyethyl)amino)-2,5-cyclohexadien-1,4-dion (HC Green No. 1), 5-hydroxy-1,4-naphthoquinone (CI75500, Natural Brown No. 7), 2-hydroxy-1,4-naphthoquinone (CI75480, Natural Orange No. 6), 1,2-dihydro-2-(1,3-dihydro-3-oxo-2H-indol-2-yliden)-3H-indol-3-on (CI73000), 4-((5-((2-hydroxyethyl)amino-1-methyl-1H-pyrazol-4-yl)imino)-4,5-dihydro-5-((2-hydroxyethyl)imino)-1-methyl-1H-pyrazol-sulfate(1:1), hydrate(1:1).

Basic dyes:

9-(dimethylamino)-benzo[a]phenoxazin-7-ium-chloride (CI51175; Basic Blue No. 6), di[4-(diethylamino)phenyl][4-(ethyl-amino)naphthyl]carbenium-chloride (CI42595; Basic Blue No. 7), di-(4-(dimethylamino)phenyl)-(4-(methyl-phenylamino)naphthalin-1-yl)carbenium-chloride (CI42563; Basic Blue No. 8), 3,7-di(dimethylamino)phenothiazin-5-ium-chloride (CI52015 Basic Blue No. 9), di[4-(dimethylamino)phenyl][4-(phenyl-amino)naphthyl]carbenium-chloride (CI44045; Basic Blue No. 26), 2-[(4-(ethyl(2-hydroxyethyl)amino)phenyl)azo]-6-methoxy-3-methyl-benzothiazolium-methylsulfate (CI11154; Basic Blue No. 41), 8-amino-2-brom-5-hydroxy-4-imino-6-[(3-(trimethylammonio)phenyl)amino]-1(4H)-naphthalinon-chloride (CI56059; Basic Blue No. 99), bis[4-(dimethylamino)phenyl][4-(methylamino)phenyl]carbenium-chloride (CI42535; Basic Violet No. 1), Tri(4-amino-3-methylphenyl)carbenium-chloride (CI42520; Basic Violet No. 2), Tris[4-(dimethylamino)phenyl]carbenium-chloride (CI42555; Basic Violet No. 3), 2-[3,6-(diethylamino)dibenzopyranium-9-yl]-benzoic acidchloride (CI45170; Basic Violet No. 10), di(4-aminophenyl)(4-amino-3-methylphenyl)carbeniumchloride (CI42510 Basic Violet No. 14), 1,3-bis[(2,4-diamino-5-methylphenyl)azo]-3-methylbenzol (CI21010; Basic Brown No. 4), 1-[(4-aminophenyl)azo]-7-(tri-methylammonio)-2-naphthol-chloride (CI12250; Basic Brown No. 16), 3-[(4-amino-2,5-dimethoxyphenyl)azo]-N,N,N-trimethyl-benzeneaminiumchloride (CI112605, Basic Orange No. 69), 1-[(4-amino-2-nitrophenyl)azo]-7-(trimethylammonio)-2-naphthol-chloride, 1-[(4-amino-3-nitrophenyl)azo]-7-(trimethylammonio)-2-naphthol-chloride (CI12251; Basic Brown No. 17), 3,7-diamino-2,8-dimethyl-5-phenylphenazinium-chloride (CI50240; Basic Red No. 2), 1,4-dimethyl-5-[(4-(dimethylamino)phenyl)azo]-1,2,4-triazolium-chloride (CI11055; Basic Red No. 22), 2-hydroxy-1-[(2-methoxyphenyl)azo]-7-(trimethylammonio)-naphthalin-chloride (CI12245; Basic Red No. 76), 2-[2-((2,4-dimethoxyphenyl)amino)ethenyl]-1,3,3-trimethyl-3H-indol-1-ium-chloride (CI48055; Basic Yellow No. 11), 3-methyl-1-phenyl-4-[(3-(trimethylammonio)phenyl)azo]-pyrazol-5-one-chloride (CI12719; Basic Yellow No. 57), di[4-(dimethylamino)phenyl]iminomethan-hydrochloride (CI41000; Basic Yellow No. 2), bis[4-(diethylamino)phenyl]phenylcarbenium-hydrogensulfate (1:1) (CI42040; Basic Green No. 1), di(4-(dimethylamino)phenyl)-phenylmethanol (CI42000; Basic Green No. 4), 1-(2-morpholiniumpropylamino)-4-hydroxy-9,10-anthraquinone-methylsulfate, 1-[(3-(dimethyl-propylaminium)propyl)amino]-4-(methylamino)-9,10-anthraquinone-chloride.

Neutral Azo Dyes:

1-[di(2-hydroxyethyl)amino]-3-methyl-4-[(4-nitrophenyl)azo]-benzene (CI11210, Disperse Red No. 17), 1-[di(2-hydroxy-ethyl)amino]-4-[(4-nitrophenyl)azo]-benzene (Disperse Black No. 9), 4-[(4-aminophenyl)azo]-1-[di(2-hydroxyethyl)amino]-3-methylbenzene (HC Yellow No. 7), 2,6-diamino-3-[(pyridin-3-yl)azo]-pyridine, 2-((4-(acetylamino)phenyl)azo)-4-methyl-phenene (CI11855; Disperse Yellow No. 3).

Acidic Dyes:

6-hydroxy-5-[(4-sulfophenyl)azo]-2-naphthaline-sulfonic acid-disodium salt (CI15985; Food Yellow No. 3; FD&C Yellow No. 6), 2,4-dinitro-1-naphthol-7-sulfonic acid-disodium salt (CI10316; Acid Yellow No. 1; Food Yellow No. 1), 2-(Indan-1,3-dion-2-yl)chinoline-x,x-sulfonic acid (mixture of mono- and disulfonic acid) (CI47005;D&C Yellow No. 10; Food Yellow No. 13; Acid Yellow No. 3), 5-hydroxy-1-(4-sulfophenyl)-4-[(4-sulfophenyl)azo]pyrazol-3-carboxylic acid-trisodium salt (CI19140; Food Yellow No. 4; Acid Yellow No. 23), 9-(2-Carboxyphenyl)-6-hydroxy-3H-xanthen-3-one (CI45350; Acid Yellow No. 73; D&C Yellow No. 8), 4-((4-amino-3-sulfo-phenyl)azo)benzenesulfonic acid-disodium salt (CI13015, Acid Yellow No. 9), 5-[(2,4-dinitrophenyl)amino]-2-phenylamino-benzenesulfonic acid-sodium salt (CI10385; Acid Orange No. 3), 4-[(2,4-dihydroxyphenyl)azo]-benzenesulfonic acid monosodium salt (CI14270; Acid Orange No. 6), 4-[(2-hydroxynaphth-1-yl)azo]-benzenesulfonic acid-sodium salt (CI15510; Acid Orange No. 7), 4-[(2,4-dihydroxy-3-[(2,4-dimethylphenyl)azo]-phenyl)azo]-benzenesulfonic acid-sodium salt (CI20170; Acid Orange No. 24), 4-hydroxy-3-[(4-sulfonaphth-1-yl)azo]-1-naphthaline-sulfonic acid-disodium salt (CI14720; Acid Red No. 14), 4-hydroxy-3-[(2-methoxyphenyl)azo]-1-naphthaline-sulfonic acid-monosodium salt (CI14710; Acid Red No. 4), 6-hydroxy-5-[(4-sulfonaphth-1-yl)azo]-2,4-naphthaline-disulfonic acid-trisodium salt (CI16255; Ponceau 4R; Acid Red No. 18), 3-hydroxy-4-[(4-sulfonaphth-1-yl)azo]-2,7-naphthaline-disulfonic acid-trisodium salt (CI16185; Acid Red No. 27), 8-amino-1-hydroxy-2-(phenylazo)-3,6-naphthaline-disulfonic acid-disodium salt (CI17200; Acid Red No. 33), 5-(acetylamino)-4-hydroxy-3-[(2-methylphenyl)azo]-2,7-naphthaline-disulfonic acid-disodium salt (CI18065; Acid Red No. 35), 2-(3-hydroxy-2,4,5,7-tetraiod-dibenzopyran-6-on-9-yl)-benzoic acid-disodium salt (CI45430; Acid Red No. 51), N-[6-(diethylamino)-9-(2,4-disulfophenyl)-3H-xanthen-3-yliden]-N-ethylethanammonium-hydroxide, internal salt, Sodium salt (CI45100; Acid Red No. 52), 8-[(4-(Phenylazo)phenyl)azo]-7-naphthol-1,3-disulfonic acid-disodium salt (CI27290; Acid Red No. 73), 2′,4′,5′,7′-tetrabrom-3′,6′-dihydroxyspiro[isobenzofuran-1(3H),9′-[9H]xanthen]-3-on-disodium salt (CI45380 Acid Red No. 87), 2′,4′,5′,7′-tetrabrom-4,5,6,7-tetrachlor-3′,6′-dihydroxyspiro[isobenzofuran-1(3H),9′ [9H]xanthen]-3-on-disodium salt (CI45410; Acid Red No. 92), 3′,6′-dihydroxy-4′,5′-diiodospiro[isobenzofuran-1(3H),9′(9H)-xanthen]-3-on-disodium salt (CI45425; Acid Red No. 95), 2-hydroxy-3-((2-hydroxynaphth-1-yl)azo)-5-nitrobenzenesulfonic acid-monosodium salt (CI15685; Acid Red No. 184), (2-sulfophenyl)di[4-(ethyl((4-sulfophenyl)methyl)amino)phenyl]-carbenium-disodium salt betaine (CI42090; Acid Blue No. 9; FD&C Blue No. 1), 1,4-bis[(2-sulfo-4-methylphenyl)amino]-9,10-anthraquinone-disodium salt (CI 61570; Acid Green No. 25), bis[4-(dimethylamino)phenyl]-(3,7-disulfo-2-hydroxynaphth-1-yl)carbenium-internal salt monosodium salt (CI44090; Food Green No. 4; Acid Green No. 50), bis[4-(diethylamino)phenyl](2,4-disulfophenyl)carbenium-internal salt, sodium salt (2:1) (CI42045; Food Blue No. 3; Acid Blue No. 1), bis[4-(diethylamino)phenyl](5-hydroxy-2,4-disulfophenyl)carbenium-internal salt, calcium salt (2:1) (CI42051; Acid Blue No. 3), 1-amino-4-(cyclohexylamino)-9,10-anthraquinone-2-sulfonic acid-sodium salt (CI62045; Acid Blue No. 62), 1-amino-4-(phenylamino)-9,10-anthraquinone-2-sulfonic acid (CI62055; Acid Blue No. 25), 2-(1,3-dihydro-3-oxo-5-sulfo-2H-indol-2-yliden)-2,3-dihydro-3-oxo-1H-indol-5-sulfonic acid-disodium salt (CI73015; Acid Blue No. 74), 9-(2-Carboxyphenyl)-3-[(2-methylphenyl)amino]-6-[(2-methyl-4-sulfo-phenyl)amino]xanthylium-internal salt, monosodium salt (CI45190; Acid Violet No. 9), 1-hydroxy-4-[(4-methyl-2-sulfophenyl)amino]-9,10-anthraquinone-sodium salt (CI60730; D&C Violet No. 2; Acid Violet No. 43), bis[3-nitro-4-[(4-phenylamino)-3-sulfo-phenylamino]-phenyl]-sulfone (CI10410; Acid Brown No. 13), 5-amino-4-hydroxy-6-[(4-nitrophenyl)azo]-3-(phenylazo)-2,7-naphthaline-disulfonic acid disodium salt (CI20470; Acid Black No. 1), 3-hydroxy-4-[(2-hydroxynaphth-1-yl)azo]-7-nitro-1-naphthaline-sulfonic acid-chromium complex (3:2) (CI15711; Acid Black No. 52), 3-[(2,4-dimethyl-5-sulfophenyl)azo]-4-hydroxy-1-naphthaline-sulfonic acid-disodium salt (CI14700; Food Red No. 1; Ponceau SX; FD&C Red No. 4), 4-(acetylamino)-5-hydroxy-6-[(7-sulfo-4-[(4-sulfophenyl)azo]naphth-1-yl)azo]-1,7-naphthaline-disulfonic acid-tetrasodium salt (CI28440; Food Black No. 1), 3-hydroxy-4-(3-methyl-5-oxo-1-phenyl-4,5-dihydro-1H-pyrazol-4-ylazo)-naphthaline-1-sulfonic acid-sodium salt, chromium complex (Acid Red No. 195).

Further colorants known for the purpose of hair coloration, which may be contained in the coloring composition in accordance with the present invention are disclosed in E. Sagarin, “Cosmetics, Science and Technology”, Interscience Publishers Inc., New York (1957), page 503 and onwards, as well as H. Janistyn, “Handbuch der Kosmetika und Riechstoffe”, Volume 3 (1973), page 388 and onwards and K. Schrader, “Grundlagen und Rezepturen der Kosmetika”, 2nd Edition (1989), pages 782-815.

Suitable hair coloring pigments are substantially insoluble colorants in the medium employed in the composition of the present invention and these pigments may be inorganic or organic. Inorganic-organic mixed pigments are also suitable. The pigments are preferably not nanopigments. The preferred particle size is from 1 to 200 μm, preferably 3 to 150 μm, more preferably 10 to 100 μm. Preferred are inorganic pigments. The inorganic pigments may be of natural origin, and may for example be prepared from chalk, ochre, umbra, green earth, burnt terra di sienna or graphite. The pigments may be white pigments, such as titanium dioxide or zinc oxide, they may be black pigments, such as iron oxide black, they may be colored pigments, such as ultra marine or iron oxide red, they may be gloss pigments, metallic effect pigments, pearlescent pigments as well as fluorescent or phosphorescent pigments, although preferably at least one pigment is a colored pigment, i.e. a non-white pigment. Suitable are metal oxides, metal hydroxides and metal oxide hydrates, mixed phase pigments, sulphur-containing silicates, metal sulphides, complex metal cyanides, metal sulphates, metal chromates and metal molybdates as well as the metals themselves (bronze pigments). Suitable are in particular titanium dioxide (CI 77891), black iron oxide (CI 77499), yellow iron oxide (CI 77492), red and brown iron oxide (CI 77491), manganese violet (CI 77742), ultramarine (sodium-aluminium sulfosilicates, CI 77007, pigment blue 2), chromium oxide hydrate (CI 77289), iron blue (ferric ferrocyanide, CI 77510), carmine (cochineal). In particular preferred are pigments on the basis of mica or laminated silica minerals which are coated with a metal oxide or a metal oxychloride, such as titanium dioxide or bismuth oxychloride, as well as additional coloring components, such as iron oxides, iron blue, ultramarine, carmine, etc., wherein the color is determined by means of the variation of the layer thickness. Such pigments are for example sold under the trade names Rona®, Colorona®, Dichrona® and Timiron® of the Company Merck, Germany. Organic pigments are for example the natural pigments sepia, gummigutt, bone coal, cassel brown, indigo, chlorophyll and other plant pigments. Synthetic organic pigments are for example azo pigments, anthraquinoid pigments, indigoid pigments, dioxazine pigments, chinacridone pigments, pthalocyanine pigments, isoindolinone pigments, perylene pigments, perinone pigments, metal complex pigments, alkali blue pigments and diketopyrrolopyrrol pigments.

The hair treatment composition in accordance with the present invention preferably comprises additionally at least one hair cosmetic in an amount of from 0.01 to 10, more preferably of from 0.05 to 5 weight %. Preferred hair cosmetics are silicone compounds as well as cationic compounds, which possess due to cationic groups or groups which can be rendered cationic, in particular primary, secondary, tertiary or quaternary amine groups, a substantivity to human hair. Suitable cationic compounds are selected among cationic surfactants, betainic, ampoteric surfactants, cationic polymers, silicone compounds having a cationic group or groups which can be rendered cationic, cationic derivatized proteins or protein hydrolyzates and betain.

Suitable silicone compounds are for example polydimethylsiloxane (INCI: Dimethicone), α-hydro-ω-hydroxypolyoxydimethylsilylene (INCI: Dimethiconol), cyclic dimethylpolysiloxane (INCI: cyclomethicone), trimethyl (octadecyloxy) silane (INCI: stearoxytrimethylsilane), dimethylsiloxane/glycol copolymer (INCI: dimethicone copolyol), dimethylsiloxane/aminoalkylsiloxane copolymer having terminal hydroxy groups (INCI: amodimethicone), monomethylpolysiloxane having lauryl side chains and terminal polyoxyethylene chains and/or polyoxypropylene chains (INCI: lauryl methicone copolyol), dimethylsiloxane/glycol copolymer acetate (INCI: dimethiconcopolyol acetate), dimethylsiloxane/aminoalkylsiloxane copolymer having terminal trimethylsilyl groups (INCI: trimethylsilylamodimethicone). Preferred silicone polymers are dimethicone, cyclomethicone and dimethiconole. Mixtures of silicone polymers are also suitable, for example a mixture of dimethicone and dimethiconol. The designations provided in brackets correspond to the nomenclature in accordance with INCI (International Cosmetic Ingredients), which is employed for the designation of cosmetically relative active ingredients and additives.

Usually further known cosmetic additives may be added to the hair treatment composition in accordance with the present invention, for example non-fixative non-ionic polymers, such as polyethylene glycols, non-fixative, anionic and natural polymers as well as mixtures thereof, in amounts of preferably from 0.01 to 50 weight %. Perfume oils in amount of from 0.01 to 5 weight %, opacifier such as ethyleneglycoldistearate in an amount of from 0.01 to 5 weight %, surfactants or emulsifiers selected from the classes of anionic, cationic, amphoteric or non-ionic surfactants, such as fatty alcohol sulfates, ethoxylated fatty alcohols, fatty acid alcanolamides, such as esters of hydrated fatty acids from rizinus oil, in an amount of from 0.1 to 30 weight %, as well as moisturizers, dye receptivity improving agents, light protective agents, anti-oxidative agents as well as preservatives in amounts of from 0.01 to 10 weight % may be added.

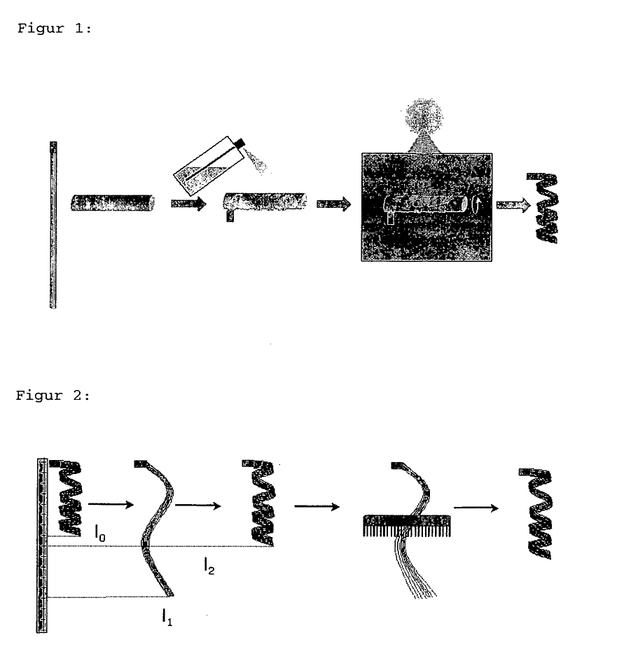

FIG. 1 shows schematically the method for the provision of a recallable, permanent hairdo. A strand of hair is wound onto a curler (bobbin) and a solution in accordance with the present invention comprising a cross-linkable macromer is sprayed thereon. By irradiation with a suitable source of energy, for example a UV lamp, the desired permanent shade is fixed. Subsequently the curler is removed.

FIG. 2 shows the deformation of a permanent hairdo and the recovery of the permanent shape starting from the temporary shape. The hair curl in the permanent shape possesses a length l₀. The curl in the deformed shape has a length l₁. The curl in the recovered shape has a length l₂. The degree of re-attaining the permanent shape (recovery) can be calculated as follows: Recovery=(l₁−l₂)/(l₁−l₁)

As measure for the evaluation of the shape memory property of a composition the memory factor may be employed, which considers the transformation ability of a permanent hairdo into a temporary shape (shaping factor) as well as the recovery attainability of the permanent form starting from the temporary form (recovery factor, degree of recovery). If one starts with the consideration from an even strand of hair onto which a curly shape has been impressed as permanent shape and onto which, subsequently, a second, even shape has been impressed as temporary shape, the shaping factor can be determined in accordance with the following criterion:

| Degree of evenness | Shaping factor |
|---|---|
| Strong curl from the hairline to the end of hair | 0 |
| Lesser degree of curliness from the hairline to | 1 |
| the end of hair | |
| Hairline even, end of hair in the form of a curl | 2 |
| Hairline even, slight curvature at the end of hair | 3 |
| Even from the end of hairline to the end of hair | 4 |

The recovery factor can be determined in accordance with the following criterion:

| Degree of recovery of the permanent shape | Recovery factor |
|---|---|
| 0% | 0 |
| 30% | 1 |
| 40% | 2 |
| 50% | 3 |
| 60% | 4 |
| 75% | 5 |
| 100% | 6 |

The memory factor M can be calculated using the shaping factor F, the maximum shaping factor F=4, the recovery factor r and the maximum recovery factor R=6 in accordance with the following equation
M=(f/F)*(r/R)*100

The memory factor should ideally be not below 25, preferably the factor should be between 25 and 33.3, more preferred between 37 and 100.

The following examples are intended to illustrate the subject matter of the present invention further.

### EXAMPLES 1

### Hair Treatment using a Thermoset Shape Memory Polymer

| | A | |
|---|---|---|
| Poly(ε-caprolactone dimethacrylate¹) | 2 | g |
| THF | Ad 100 | g |

¹Prepared in accordance with Example 2 of WO 99/42147

Onto even hair strands having each a length of 19 cm approximately 1 g of the composition are applied. The strands are wound on curlers and dried. Subsequently the strands are warmed to about 65 to 700C and a photochemical cross-linking reaction is carried out (UV light, Hg-lamp). After cross-linking and cooling to room temperature the curlers are removed. The curls (impressed permanent shape) had a length of 8 cm.

In order to simulate a deformation by means of cold shaping the curls were subjected to a load of 25 g and were let to hang at room temperature for a) 1 hour, b) 2 hours and 3) 3 hours. The length of the curls after deformation were a) 10 cm, b) 10 cm and c) 11.2 cm.

After removal of the weights the strands were heated to about 65 to 70° C. The curls retracted spontaneously to lengths of a) 8 cm, b) 8 cm and c) 8.3 cm. This corresponds to a degree of recovery of the impressed hairdo of a) 100%, b) 100% and c) 91%. The degree of recovery (Recovery) can be calculated (see FIG. 2) as follows:
Recovery=(1₁−1₂)/(1₁−1₀)

In order to impress a second hairdo (temporary shape) a wavy strand (curl) of a length of 3.2 cm was warmed to 70° C. and stretched to the initial, complete length of 6.3 cm. Subsequently the strand was cooled. Upon heating to about 65 to 70° C. the curl spontaneously retracted to a length of 3.9 cm. This corresponds to a degree of recovery of the impressed hairdo (shape) of 77%.

### Examples 2 To 31

### Hair Treatment Using a Shape Memory Polymer

Compositions were prepared comprising each 2 weight % macromer in a mixture of ethanol and water (50/50) employing the following macromers and macromer mixtures in the indicated weight ratios.

| | 2 | 3 | 4 | 5 | 6 | 7 | 8 | 9 |
|---|---|---|---|---|---|---|---|---|
| PEG(4k)-DMA | 100 | 100 | | | | | | |
| PEG(8k)-DMA | | | 100 | 100 | 70 | 70 | 80 | 80 |
| PPG-(475)-MA | | | | | 30 | 30 | 20 | 20 |
| initiator | — | AiBN | — | AiBN | — | AiBN | — | AiBN |

| | 10 | 11 | 12 | 13 | 14 | 15 | 16 | 17 |
|---|---|---|---|---|---|---|---|---|
| PEG(8k)-DMA | 70 | 70 | 80 | 80 | 70 | 70 | 80 | 80 |
| PEG-(526)-MA | 30 | 30 | 20 | 20 | | | | |
| PPG-(430)-MA | | | | | 30 | 30 | 20 | 20 |
| initiator | — | AiBN | — | AiBN | — | AiBN | — | AiBN |

| | 18 | 19 | 20 | 21 | 22 | 23 | 24 | 25 |
|---|---|---|---|---|---|---|---|---|
| PEG(8k)-DMA | 70 | 70 | 80 | 80 | 70 | 70 | 80 | 80 |
| NoO-PPG(400)-MA | 30 | 30 | 20 | 20 | | | | |
| MeO-PEG(300)-MA | | | | | 30 | 30 | 20 | 20 |
| initiator | — | AiBN | — | AiBN | — | AiBN | — | AiBN |

| | 26 | 27 | 28 | 29 | 30 | 31 |
|---|---|---|---|---|---|---|
| PEG(8k)-DMA | 70 | 70 | 80 | 80 | | |
| MeO-PPG(200)-MA | 30 | 30 | 20 | 20 | | |
| PEG(10k)-DMA | | | | | 100 | 100 |
| Initiator | — | AiBN | — | AiBN | — | AiBN |

Application of compositions 2 to 31 was carried out as explained in Example 1. The results obtained were comparable.

### Examples 32 To 41

### Hair Treatment Using Shape Memory Polymers

Compositions were prepared comprising each 2 weight % macromer in THF using the following macromers and macromer mixtures in the weight ratios as indicated:

| | 32 | 33 | 34 | 35 | 36 | 37 | 38 | 39 | 40 | 41 |
|---|---|---|---|---|---|---|---|---|---|---|
| PLGA(7k)-DMA | 100 | 100 | | | | | | | | |
| PCl(10k)-DMA | | | 100 | 100 | 70 | 70 | 70 | 70 | 70 | 70 |
| NoO-PPG(400)-MA | | | | | 30 | 30 | | | | |
| NoO-PPG(475)-MA | | | | | | | 30 | 30 | | |
| PEG(526)-MA | | | | | | | | | 30 | 30 |
| initiator | — | AiBN | — | AiBN | — | AiBN | — | AiBN | — | AiBN |

The application of compositions 32 to 41 was carried out as disclosed in Example 1. The results obtained were comparable.

### Example 42

### Hair Treatment Using Shape Memory Polymer

A composition was prepared comprising 2 weight % PLGA(7k)-DMA macromer in ethylacetate and this composition was applied on hair. After the hair has been brought into the desired shape this shape was fixed by means of irradiation with UV light.

The preparation of the macromers employed in Examples 2 to 42 was carried out on the basis of the disclosure in WO 99/42147.

The abbreviations as used in the examples to designate the following materials:

PEG(4k)-DMA, PEG(8k)-DMA, PEG(10k)-DMA: poly(ethylene-glycol)-dimethacrylate

PPG-(475)-MA, PPG-(430)-MA: poly(propylene-glycol)-methacrylate

PEG-(526)-MA poly(ethylene-glycol)-methacrylate

NoO-PPG(400-MA, NoO-PPG(475)-MA: nonyl-poly(propylene-glycol)-methacrylate

MeO-PEG-(300)-MA: methyl-poly(ethylene-glycol)-methacrylate

MeO-PPG(200)-MA: methyl-poly(propylene-glycol)-methacrylate

PLGA(7k)-DMA: poly(L-lactide-co-glycolide)-dimethacrylate

PC1(10k)-DMA: poly(ε-caprolactone)-dimethacrylate

1. A method for hair treatment, comprising the steps of
applying a composition which comprises at least one cross-linkable macromer, which forms after crosslinking a shape-memory polymer, onto hair, wherein the macromer
a) comprises cross-linkable segments, which are cross-linkable by means of chemical bonds, and
b) thermoplastic segments, which are not chemically cross-linkable,
previously, at the same time or subsequently bringing the hair into a desired (permanent) shape, and,
subsequently fixing the shape by means of chemical cross-linking of the macromer in order to form the shape-memory polymer,
wherein the shape-memory polymer possesses at least one transition temperature Tₜᵣₐₙₛ.

2. A method for hair treatment, wherein
a hairdo (permanent shape) programmed in accordance with the method according to claim 1 is heated to a temperature above Tₜᵣₐₙₛ;
the hair is brought into a second (temporary) shape; and
the second shape is fixed by means of cooling to a temperature below Tₜᵣₐₙₛ.

3. A method for the recovery of a programmed hairdo (permanent shape) obtained in accordance with the method of claim 1, wherein a hairdo in a temporary shape according to claim 2 or a hairdo deformed by means of cold forming, is heated to a temperature above Tₜᵣₐₙₛ.

4. A method in accordance with any claim 1, wherein the cross-linkable macromer is selected among compounds having the general formula
A1-(X)ₙ-A2 (I)
wherein A1 and A2 represent a reactive, chemically cross-linkable group and wherein —(X)ₙ— represents a divalent, thermoplastic polymer segment or oligomer segment.

5. A method according to claim 4, wherein the cross-linkable macromer is selected from polyesters, oligoesters, polyalkylene-glycols, olygoalkylene-glycols, polyalkylene carbonates and oligoalkylene carbonates substituted with at least two acrylate groups or methacrylate groups.

6. A method in accordance with claim 5, wherein the cross-linkable macromer is selected among poly(ε-caprolactone)-dimethacrylate, poly(DL-lactide)-dimethacrylate, poly(L-lactide-co-glycolide)-dimethacrylate, poly(ethylene-glycol)dimethacrylate, poly(propylene-glycol)dimethacrylate, PEG-block-PPG-block-PEG-dimethacrylate, poly(ethyleneadipate)-dimethacrylate and hexamethylenecarbonatedimethacrylate.

7. A method in accordance with claim 1, wherein the composition comprises in addition a macromer having only one chemically reactive group, provided at the terminal or at a side chain.

8. A method in accordance with claim 7, characterized in that the additional macromer is selected among compounds of the following general formula
R—(X′)ₙ-A3
wherein R represents a monovalent organic residue, A3 represents a reactive, chemically cross-linkable group and wherein —(X′)ₙ— represents a divalent, thermoplastic polymer segment or oligomer segment.

9. A method in accordance with claim 8, wherein the additional macromer is selected among polyalkylene-glycols substituted with one acrylate group or methacrylate group and monoalkylethers thereof as well as block copolymers thereof.

10. A method in accordance with claim 9, wherein the additional macromer substituted with only one chemically reactive group is selected among poly(ethylene-glycols)monoacrylate, poly(propylene-glycol)monoacrylate and monalkylethers thereof.

11. A cosmetic composition comprising in a cosmetically suitable medium at least one macromer which is cross-linkable to a shape-memory polymer, wherein the shape-memory polymer possesses at least one transition temperature Tₜᵣₐₙₛ and wherein the macromer comprises
a) cross-linkable segments which are cross-linkable by means of chemical bonds, and
b) thermoplastic segments, which are not chemically cross-linkable.

12. A composition in accordance with claim 11, wherein the cross-linkable macromer is contained in an amount of from 0.01 to 25 weight %.

13. A composition in accordance with claim 11, wherein in addition 0.01 to 25 weight % of at least one additional macromer with only one terminal or side chain chemically reactive group and/or at least one active principle, selected from hair cosmetics, hair fixatives and hair coloring agents is contained.

14. A cosmetic composition comprising a composition in accordance with claim 11, characterized in that it is provided in the form of a lotion, a spray lotion, a cream, a gel, a foam gel, an aerosol spray, a non-aerosol spray, an aerosol foam, a non-aerosol foam, a O/W-emulsion or W/O-emulsion, a macro emulsion or a hair wax.

15. A method of treating hair with macromers that are cross-linkable to shape-memory polymers, wherein the macromer comprises
a) cross-linkable segments, which are cross-linkable by means of chemical bonds, and
b) thermoplastic segments, which are not chemically cross-linkable,
wherein the shape-memory polymers possess at least one transition temperature Tₜᵣₐₙₛ.
